(19) United States
(12) Patent Application Publication
(10) Pub. No.: US 2008/0184998 A1
(43) Pub. Date: Aug. 7, 2008

(54) Foil Cutter In A Dry Power Inhaler And A Method For Cutting Open A Container

(75) Inventors: Mattias Myrman, Tyreso (SE); Mats Eriksson, Jarfalla (SE)

Correspondence Address: OBLON, SPIVAK, MCCLELLAND MAIER & NEUSTADT, P.C., 1940 DUKE STREET, ALEXANDRIA, VA, 22314, US

(73) Assignee: MEDERIO AG, Hergiswil NW (CH)

(21) Appl. No.: 11/722,254

(22) PCT Filed: Dec. 12, 2005
(86) PCT No.: PCT/SE05/01908

§ 371 Date: Jun. 20, 2007

(30) Foreign Application Priority Data
Dec. 20, 2004 (SE) 040115-9

(51) Int. Cl. A61M 15/00; B67B 7/46

(52) U.S. Cl. 128/203.15; 7/156

(57) ABSTRACT

An improved foil-cutting device is disclosed for opening a foil protecting a pre-metered dose of medication power carried by dose carrier to be used in an inhaler device. The dose carrier with the medication powder is covered by a seal, e.g. metallic or plastic foil. The foil-cutter device of the present invention provides a knife device tool for simultaneous access of a selected sealed dose of medication powder during an inhalation process using a dry powder inhaler (DPI).

### TECHNICAL FIELD

The present invention relates to a method applied in a dry powder inhaler for cutting open a sealed cover of a dose container in connection with an inhalation giving an inhalation air stream access to a metered dose of a dry powder medicament enclosed in the sealed container.

### BACKGROUND

Within health care today administration of medicaments by inhalation for distributing dry powder medicaments directly to the airways and lungs of a user is becoming more and more popular, because it offers an efficient, fast, and user friendly administration of the specific medication substance.

Different types of inhalers are available on the market today, such as metered dose inhalers (MDIs), nebulizers and dry powder inhalers (DPIs). MDIs use medicaments in liquid form and may use a pressurized drive gas to release a dose. Usually MDIs have a relatively low capacity for delivering an effective dose of the active substance in a single inhalation and many devices have problems with using a drive gas, which is environmentally acceptable. Nebulizers are fairly big, non-portable devices and are rarely used outside medical clinics. Dry powder inhalers have become more and more accepted in the medical service, because they deliver an effective dose in a single inhalation, they are reliable, often quite small in size and easy to operate for a user. Two types are common, multi-dose dry powder inhalers and single dose dry powder inhalers. Multi-dose devices have the advantage that a quantity of medicament powder, enough for a large number of doses, is stored inside the inhaler and a dose is metered from the store shortly before it is supposed to be inhaled. Single dose inhalers either require reloading after each administration or they may be loaded with a limited number of individually packaged doses, where each package is opened shortly before inhalation of the enclosed dose is supposed to take place.

For example, respiratory disorders, such as asthma and COPD, have been successfully treated by multi-dose as well as single dose inhalers. The active substances, e.g. budesonide and formoterol, are very stable and insensitive to variations in ambient conditions, so it does not matter that the powder store inside a multi-dose inhaler, such as Turbuhaler™ from AstraZeneca, is exposed to ambient air for a long time. Requirements are low regarding resistance to moisture diffusion concerning the dose packages used in single dose inhalers for this kind of substance.

Generally, doses need to be protected by an enclosure not only during storage, but also when inserted in a dry powder inhaler, where the dose and enclosure are kept in a ready state for delivery in an inhalation. It is not satisfactory if the inhaler is designed such that the user may open a dose container, to allow access to the dose therein before an inhalation commences, and then not go through with the inhalation immediately.

Gelatin or plastic capsules and blisters made of aluminum or plastic, or laminates comprising aluminum and plastic foil are common prior art containers for metered single doses of dry powder medicaments. Besides a method of breaking the container open inside the inhaler and pour out the dose, the most common method of opening the container is to punch one or more holes in the container itself or in a foil sealing the container. In the first case the powder is poured onto a surface inside the inhaler and made available for inhalation from there.

In our U.S. Pat. No. 6,651,341 (WO 02/24266) titled “Foil cutter”, the teachings of which are included in full in this document by reference, an arrangement is disclosed for cutting open a foil protecting a dry powder dose and the invention is directed towards solving some of the problems associated with single dose dry powder inhalers. However, we have found some shortcomings in the disclosed foil cutter. For instance, the cutter is not allowed to come in contact with the enclosed powder dose during a cutting operation. The dose is sealed by a foil enclosing the chosen dose in a recess of the cassette acting as a dose carrier. In our continued research, we have found that in other types of enclosures, such as dose containers, it is impossible to rule out the possibility that the cutter will come in contact with the dose powder during a cutting operation. The cutter and cutting method must preferably be adapted to account for this possibility. In a second aspect, the wedge-like form of the foil cutter creates too much friction with the foil being cut, which may result in very crumpled foil getting in the way of a suction nozzle sucking up the powder. The crumpling can go so far that bits of foil risk being torn loose and may contaminate the powder of the dose, thus constituting a risk for the user.

Therefore there is a demand for an improved method and device, which will open the dose container to make the powder dose enclosed therein easily available for an immediate access coordinated with an inhalation process for the release of a fresh dose of medication powder by the DPI.

### SUMMARY

A dose container enclosing a dry powder medicament dose will in a typical embodiment carry a pre-metered dose. The medicament powder of the dose is physically protected by a container cover that stops external influences from the ambient atmosphere and from a user from affecting the integrity of the dose until the container is opened up. In a particular embodiment the cover is in the form of a sealing foil, tightly affixed onto the container. The present invention discloses an improved method of cutting a closed dose container open and comprises the steps of first penetrating the container cover at a first point, then slitting the cover open from the first point to a second point of the cover by introducing a relative motion between a knife device, doing the penetrating and slitting operation, and the container, whereby the dose is made available for an aerosolizing stream of air, which advantageously is introduced through the slit, thereby accessing the powder in the opened container. Preferably, the first and second point of the cover are positioned at diametrically opposite sides of the container, such that the slit is long enough to uncover all of the powder in the dose for the benefit of making almost all of the powder available to an aerosolizing air-stream.

The present invention also discloses a knife device for performing the method and providing an improved tool for accessing an enclosed, sealed dose of medication powder in the container. The knife device is placed into a dry powder inhaler (DPI), such that a relative motion may be arranged between the knife and an inserted, selected dose container. Preferably, the knife device will slit the container open within fractions of a second before the uncovered powder of the dose is sucked up by an inhalation air-stream. Particularly, the present invention is adapted to be applied in an inhaler controlling the opening of a dose container and release of a dry powder dose therein during a single inhalation.

A method for opening a dose container and a knife device according to the present invention are set forth by the independent claims 1 and 11. Further embodiments of the method are set forth by the dependent claims 2 to 10 and 12 to 15.

### DESCRIPTION OF THE DRAWINGS

The invention will be described in the form of a preferred and illustrative embodiment and by means of the attached drawings, wherein like reference numbers indicate like or corresponding elements and wherein:

FIG. 1 illustrates in a flow diagram a particular method of opening a sealed container by slitting a container seal open, thereby giving access to the dose inside;

FIG. 2 illustrates in a perspective drawing a particular embodiment of a knife device, which may be used to perform the method;

FIG. 3 illustrates a side view of the knife device shown in FIG. 2;

FIG. 4 illustrates in perspective, top and side views a particular embodiment of a sealed dose container, adapted for the present invention.

FIG. 5 illustrates a typical inhalation sequence when applying the present invention to a dry powder inhaler.

### DESCRIPTION OF AN ILLUSTRATIVE EMBODIMENT

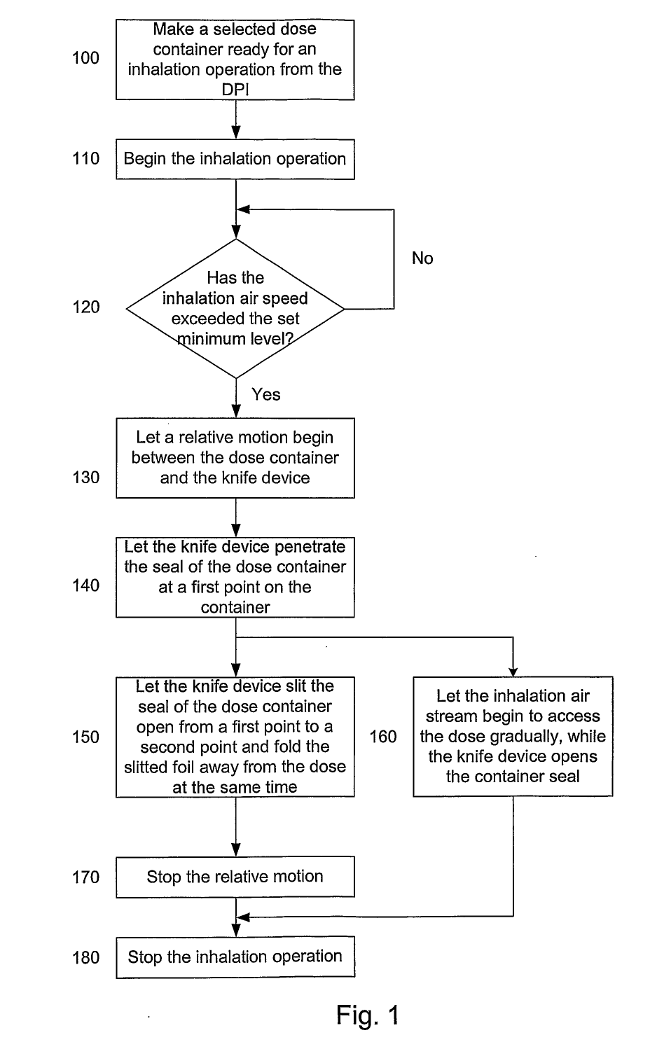

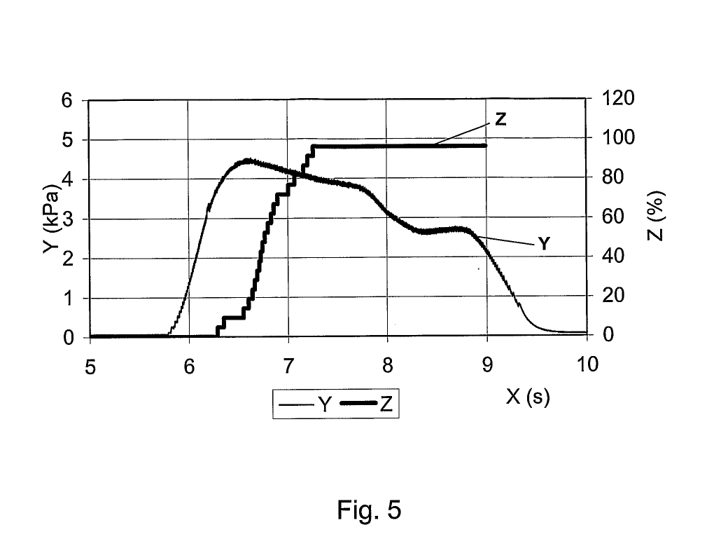

The present invention relates to an improved foil cutter—a knife device—in a dry powder inhaler (DPI) and a method of slitting open a sealed dose container, having inside a metered medicament dose of dry powder. A particular method is described in a flow chart illustrated in FIG. 1. A selected container is put in a starting position for an inhalation operation from the DPI in step 100. The inhalation operation is started in step 110, such that a suction being applied to a mouthpiece of the inhaler creates an inhalation air stream through dedicated flow channels of the inhaler. In an optional step 120 a breath-actuation device acts as a go-no-go device to release a relative motion. The relative motion between the knife device and the dose container in step 130 is optionally not released until the speed of the inhaled air stream has reached or exceeded a set minimum level. In step 140 the relative motion brings the knife device in contact with the seal at a first point on the dose container, whereby the seal is penetrated there by a point of knife. In step 150 at least one blade of the knife device slits the seal open from the point of penetration, preferably at one end of the dose container, to a second point on the container, preferably at the opposite end of the container, where the knife device exits the container seal, leaving the container opened and the slitted foil guided away from the dose inside the container. In step 160, concurrent with step 150 and 170 the stream of inhalation air is directed into the inside of the container, starting at the end of the container where the seal was first penetrated and continuing in the track made by the knife device. As a result, the air stream accesses and aerosolizes the dose powder inside the container gradually, at a pace set by the speed of the relative motion. In step 170 the relative motion is stopped at an end position, where the knife device has reached the opposite end of the container, having opened it completely. Preferably, the time for executing the opening of the dose container is in a range from 0.1 to 2 seconds and more preferably not less than 0.2 s but not more than 1.5 s. By now the air stream has moved from one end of the container to the opposite end of the container, having aerosolized all of the powder inside the container there between. In step 180 the inhalation is allowed to continue until it is complete and then stopped. See FIG. 5 which illustrates a typical opening of a container in coordinated with an inhalation. Diagram curve Y represents the suction power in kPa provided by the user over time X and curve Z represents the container motion from 0 (starting position) to 100% (end position) in the DPI.

New types of dry powder medicaments, not least for systemic treatment, have a rather short expiry date and they are generally quite sensitive to ambient conditions, especially moisture during storage and in use. Hence, the demands put on dose protection and inhaler devices in handling sensitive doses are therefore much higher than for prior art devices as used e.g. for administering traditional medicaments against respiratory disorders. For instance, prior art blister packages for dry powder medicaments, intended for inhaler use, often use a fairly thin polymeric seal, which can be easily ripped or punched open before the dose is supposed to be inhaled. Another common seal is a peelable blister such that the blister is peeled open prior to inhalation of the enclosed dose. Yet another type of prior art dose container is the capsule. Capsules are often made by gelatin, but polymers and cellulose and other materials are also used. A common problem for prior art blisters and capsules used for dry powder doses for inhalation is that they do not protect sensitive substances well enough during storage and in use from being affected by humidity.

The improved foil cutter according to the present invention may be applied to prior art containers and conventional prior art seal types, but it is developed especially for tougher dose containers and applications requiring better container seals that stop humidity and other foreign substances from entering the dose container for the shelf-life of the product.

A particular container seal is a high barrier seal, which may for instance be made up of one or more layers of materials, i.e. technical polymers, aluminum or other metals, glass, silicon oxides etc that together constitutes the high barrier seal. If the high barrier seal is a foil, a 50 μm PCTFE/PVC pharmaceutical foil is the minimum required high barrier foil if a two-week in-use stability for a moisture sensitive medicament shall be achieved. For longer in-use stabilities metal foils like aluminum foils from Alcan Singen can be used.

The foil cutter disclosed in the publication U.S. Pat. No. 6,651,341 turned out to be inadequate for slitting tough, seals, such as a high barrier seal foil. The sharp point of the cutter could penetrate the seal foil, but it could not slit the foil open without tearing the seal foil into shreds, which risked contaminating the delivered dose. A solution to this problem was the introduction of a knife device, which besides having a sharp point of knife also has a first knife-blade to make a first slit in the seal, where the blade is generally at right angles to the seal following an imaginary line on the container along which the first slit is intended to be made.

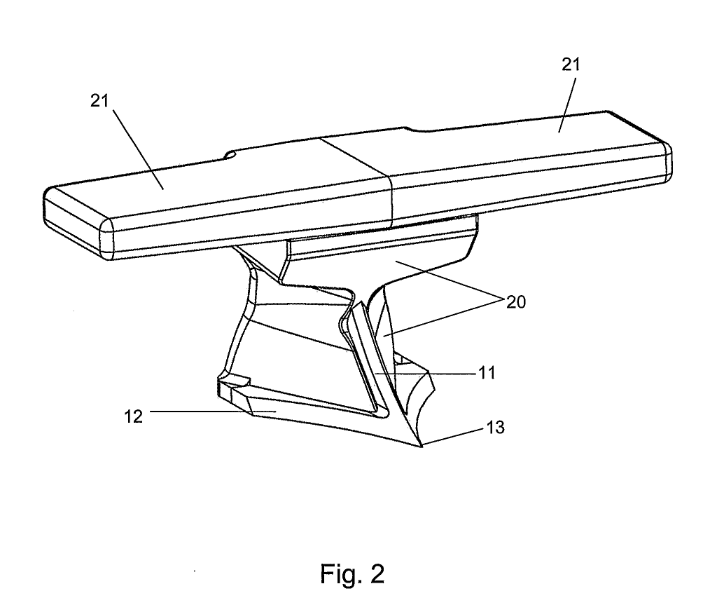

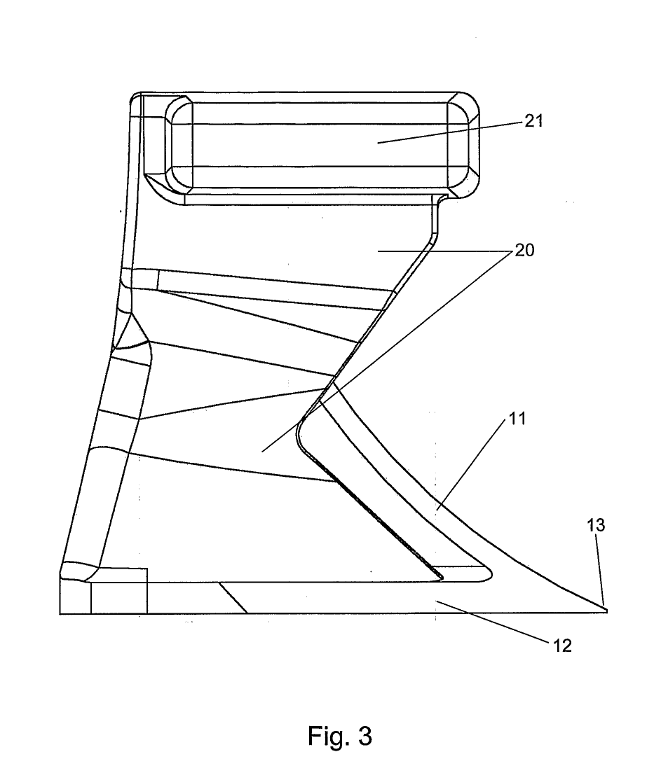

A further improvement of the prior art foil cutter is the addition of a second blade of knife. This second blade is optional to the first blade and is used to cut a second slit in addition to the first slit, made by the first knife-blade, in order to make it easier for the knife device to unfold or remove the cut seal away from the dose. The stream of inhalation air following closely in the track of the moving knife device is given much better, unhindered access to the dose inside the container if the second slit is made. This improves dose deaggregation and reduces powder retention in the container. For instance, two blades may cut out a strip of the seal and roll it up instead of unfolding the cut seal. A particular embodiment of the knife device is illustrated in FIG. 2 in a perspective drawing. The first knife-blade is designated 11 and an optional second one is designated 12. The two blades are fixed in a body 20 having two fixing flanges 21, intended for holding the knife device firmly in place inside the inhaler device. FIG. 3 illustrates the knife device from the front showing the point of knife 13. In a particular embodiment the two knife-blades are made from one piece. The blades may be positioned apart and the planes of the blades may cross at an angle from 0 to 90 degrees. The knife device illustrated in FIGS. 2 and 3 constitutes a particular embodiment, where the first knife-blade slits the seal longitudinally and the second blade cuts the seal laterally, but only at the points of entry, i.e. container penetration, and at the point of exit from the container.

The prior art foil cutter in the document cited had a further disadvantage in that as soon as the foil to be cut was made a bit more robust, thus making more resistance when being cut, the pointed knife and the knife body holding it would twist and wobble in the direction of the container dose bed. As a result, the foil was not cut cleanly, but became ragged and fragments of foil could be torn loose, which could contaminate the dose. The knife device according to the present invention reduces the friction in the cutting operation by using a sharp, dedicated first knife-blade for slitting the container seal open. The reduced friction reduces the force necessary to drive the knife device, thereby diminishing the bending force, which in turn reduces the strain on the knife device fixing flanges 21. Furthermore, the knife-blades and the body 20 holding the blades have been strengthened in the present invention to make the body and the whole knife device very rigid. The knife device of the improved foil cutter is further stiffly mounted inside the inhaler, so that bending and wobbling while cutting is eliminated. A further development of the present invention is the size and shape of the body 20, which has been improved to further reduce the friction during cutting and at the same time helping to guide the slitted foil smoothly away from the dose, without crumpling of the foil and without tearing pieces off the slitted foil.

The driving force, necessary for driving the relative motion of the knife device versus the dose container, may come from any type of power source, e.g. electric, hydraulic, pneumatic, spring, mechanical or manual by a user.

Yet another problem associated with the prior art foil cutter concerned the size and shape of the cutter, such that it tended to collect powder if the cutter came in contact with the dose powder inside the container during cutting. The cutter retained the collected powder until the next cutting operation, which risked falling off at random at a later inhalation, adding to the metered dose at that time. The knife device of the present invention is preferably designed to minimize collection of powder.

In another aspect of the present invention, the time between opening of a selected dose container and inhalation of the enclosed dose is on the order of a split second, which is so short that it is negligible. Prior art inhalers allow much longer times between subjecting the selected dose to the ambient atmosphere and an actual inhalation of the dose taking place. Some prior art inhalers have no control over time lapse between breaking the dose container open and a following inhalation. In any case, the dose may decompose rapidly by the influence of the ambient atmosphere, such that when the user finally gets round to inhaling the dose it may have deteriorated seriously. The user will then unknowingly get a smaller therapeutic effect than expected.

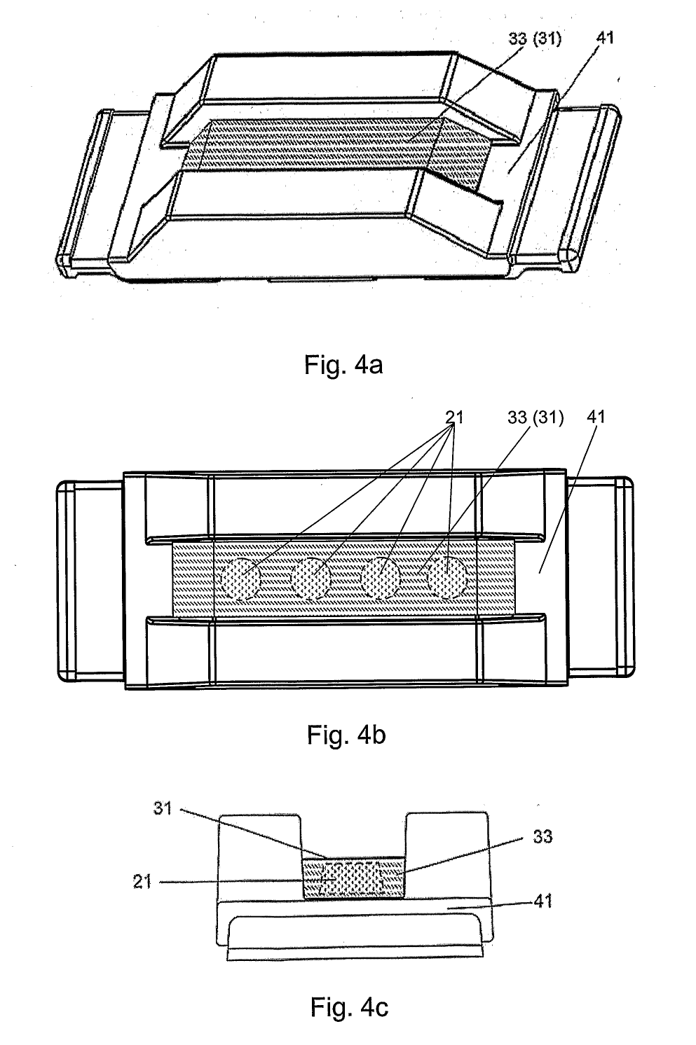

Naturally, using a new type of blister pack, a so-called pod, as a particular embodiment of a sealed dose container, is to be preferred in an application where the present invention is to be put to use. See FIG. 4 illustrating a pod carrying a sealed container in a perspective drawing. FIG. 4a shows a sealed container 33 (seal 31) put into a protective carrier 41 adapted for insertion into a dry powder inhaler. FIG. 4b shows a top view of the carrier/container and indicates depositions of dry powder making up a metered dose inside the container 33 under a seal 31, for the benefit of the reader. FIG. 4c illustrates a side view of the carrier/container in FIG. 4b.

However, the present invention may also advantageously be applied to conventional blister packs and capsules. Preferably, a person skilled in the art may use the disclosure by adapting e.g. when the container seal is to be opened during the course of moving the container from a starting position to an end position and adapting how it is opened to the particular type of container that is selected for use. Such adaptation is still within the scope of the present invention. An objective of the present invention is to make the time between opening of the container and delivery of the dose inside as short as possible and to make it impossible for the user to open the container without commencing an inhalation. If an inhalation is broken off prematurely for any reason, then the user will at least be aware that a full dosage may not have been delivered.

In cases where the medicament dosage is controlled by the user, a single dose dry powder inhaler is preferred, because the user may then select a dosage among pre-metered doses, which is well adjusted to the situation and condition the user is in.

It will be understood by those skilled in the art that various modifications and changes may be made to the present invention without departing from the scope thereof, which is defined by the appended claims.

1. A method applied in a dry powder inhaler for cutting open a sealing foil of a dose container, and commencing delivery by inhalation of a dry powder medicament dose enclosed in the container, characterized by
moving the dose container in relation to an adjacent knife device, or vice versa, said knife device having a sharp point of knife and at least one knife-edge, from a starting position to an end position inside the inhaler;
letting the sharp point of the knife device penetrate the container sealing foil at a first position and then letting the at least one knife-blade slit the foil open in a clean cut from the first position to a second position, thereby opening the container whereby the powder medicament dose in the container is exposed;
arranging a concentrated inhalation airflow close to the dose in the container in direct connection with an opening of the container sealing foil.

2. The method according to claim 1, characterized by
arranging the knife device and the sealed dose container in the inhaler such that while the sealing foil is being slit open, the slitted foil is guided away from the enclosed dose, as the knife device moves in a predefined path relative the container.

3. The method according to claim 1, characterized by
arranging the sealing foil of the container to be generally flat, and
moving the knife device in a linear motion in relation the container, or vice versa, to slit the foil open.

4. The method according to claim 1, characterized by
arranging the sealing foil of the container to be curved, and
moving the knife device in a continuous motion in relation to the container, or vice versa, to slit the foil open.

5. The method according to claim 1, characterized by
adapting the knife device for a sealing foil selected from a group of materials comprising polymers including thermoplastics, metals including aluminum, glass, ceramics or gelatine or combinations of such materials.

6. The method according to claim 1, characterized by
adapting the knife device for a sealing foil having high barrier seal properties, and
applying the foil to an open receptacle of a high barrier seal container, the receptacle carrying a metered dose, thereby obtaining a high barrier sealed container.

7. The method according to claim 1, characterized by
making the container and seal as a unitary construction using the same materials, and
adapting the knife device for the selected sealing foil material chosen from a group comprising polymers including thermoplastics, metals including aluminum, glass, ceramics or gelatine or combinations of such materials.

8. The method according to claim 1, characterized by
incorporating a second knife-blade in the knife device for improving access to the dose for the concentrated air stream, when the seal is slit open.

9. The method according to claim 1, characterized by
commencing an inhalation process through the inhaler, thereby creating an air stream, before the container seal is being slit open.

10. The method according to claim 1, characterized by slitting open the container seal before commencing an inhalation process through the inhaler.

11. A knife device applied in a dry powder inhaler for cutting open a sealing foil of a dose container, making delivery possible of an enclosed dry powder medicament directly from the container, characterized in that
the knife device comprises at least one knife-blade having a sharp point of knife and at least one knife-edge, and
the sharp point of knife and at least one knife-edge can move in a relative motion vis-à-vis the container, whereby the sharp point penetrates the sealing foil at a first position and the at least one knife-edge slits the foil open in a clean cut from the first position to a second position on the container, whereby the container is opened up exposing the powder medicament dose therein.

12. The knife device according to claim 11, characterized in that
a body of the knife device holding the sharp point and the at least one knife-blade is rigid and stiffly mounted inside the inhaler to produce a clean cut of the seal without wobbling.

13. The knife device according to claim 11, characterized in that
a second knife-blade in the knife device is incorporated for improving access to the dose for the concentrated air stream, when the seal is slit open.

14. The knife device according to claim 13, characterized in that
the second knife-blade is positioned in a plane at an angle from 0 to 90 degrees relative the plane of the first knife-blade for assisting the cutting and opening of the container seal.

15. The knife device according to claim 13, characterized in that
the first and the second knife-blades are made from a single piece of solid material.

16. The knife device according to claim 13, characterized in that
the first and the second knife-blades are made from two different pieces of solid material, which are joined together to form the knife device.
